(19) United States
(12) Patent Application Publication
(10) Pub. No.: US 2008/0247979 A1
(43) Pub. Date: Oct. 9, 2008

(54) Use of a Hydrophobic Zeolite, that Contains and Gives a Slow Release of an Active Element, for Treatment of Skin

(76) Inventors: Hakan Benjaminson, Stockholm (SE); Stan Anderson, Lottorp (SE)

Correspondence Address: CONNOLLY BOVE LODGE & HUTZ LLP, 1875 EYE STREET, N.W., SUITE 1100, WASHINGTON, DC, 20036, US

(21) Appl. No.: 12/065,600

(22) PCT Filed: Mar. 13, 2006
(86) PCT No.: PCT/SE2006/000317

§ 371 Date: Mar. 3, 2008

(30) Foreign Application Priority Data
Mar. 14, 2005 (SE) 0500564-0

(51) Int. Cl. A61K 47/02; A61P 17/00; A61K 33/18; A61K 33/40; A61K 31/05; A61K 31/045; A61Q 15/00

(52) U.S. Cl. 424/68; 514/770; 424/667; 424/616; 514/731; 514/724

(57) ABSTRACT

The invention describes the use of a hydrophobic zeolite, that contains an active component, especially a disinfection element, as ethanol, iodine, phenol, cresol or hydrogen peroxide, in a composition for non-medical treatment of the skin, for example as a deodorant. The invention also describes the use of a hydrophobic zeolite, that contains an active component, especially a disinfection element, as ethanol, iodine, phenol, cresol or hydrogen peroxide, for manufacturing of a pharmaceutical preparation for treatment of skin related conditions and diseases, as skin infections. The hydrophobic zeolite is especially selected from that group that comprises silicalite, or hydrophobic ZMS-5, hydrophobic mordenite and hydrophobic zeolite Y.

### TECHNICAL AREA

The invention is related to a new use of a hydrophobic zeolite, which contains and gives a slow release of an active component. More specifically, the invention relates to a hydrophobic zeolite used for treatment of the skin.

### BACKGROUND OF THE INVENTION

For treatment of different skin related conditions and diseases, for example skin infections, it is known to use different kinds of elements with disinfection qualities. Some well known elements with disinfection qualities are for example, ethanol, iodine, phenol, cresol and hydrogen peroxide. It is known that these elements, amongst many, are included in preparations of powders, fluids, ointments, or bandages in order to achieve a bacteria killing effect on the skin. Ethanol is eagerly used because of its good effect with few or no side effects. Normally a water solution is used, that contains 60-70 weight percent ethanol which is put directly on wounds in order to counteract the beginning of an infection, or to damp ongoing infection. The ethanol evaporates however, quickly and more difficult infections, which can be lying deeply, or fungus infections, remains in the long run unaffected by the ethanol mixture. It would therefore be desirable to achieve a composition with a slow release of an active element, for example ethanol, to thereby give a more effective treatment of the skin related conditions and diseases.

It is known to use hydrophilic zeolites in combination with an active element. Hydrophilic zeolites shall not be mixed up with hydrophobic zeolites, since they differ in both structures and functions.

Hydrophilic zeolites are water containing sodium aluminium silicalites. Hydrophilic zeolite Y has a typical composition, Na₂Al₂Si₅O₁₄·10H₂O, while hydrophilic zeolite A has a typical composition of NaAlSiO₄·2H₂O. They are both particular effective as dehydrators and both are also good ion exchangers. For a hydrophilic zeolite Y, it is necessary that it forcefully binds water, and this water is bounded to the Na-ions. It is possible to bind an organic molecule to a hydrophilic zeolite, for example hydrophilic zeolite Y, but this does require extensive measures and does not lead to a composition with a slow release of organic molecules. Thus the water must first be eliminated from the zeolite, for example through a vacuum treatment and then kept in a dry-box. Then the organic molecules can be introduced in to the hydrophilic zeolite. After this procedure when the zeolite is put out into the normal atmosphere the hydrophilic properties makes it absorb water fast, which push out the organic molecules, that leaves the zeolite quickly. These properties make the hydrophilic zeolites inappropriate for achieving a composition with a slow release off an organic element.

Below are some examples of publications given, that describes the use of hydrophilic zeolites.

DD 278 494 A1 describes a bandage for a regulated free release off a pharmacologist active element, for example antibiotic. The bandage includes a hydrophilic zeolite, where the antibiotic is adsorbed.

RU 2083224 C1 describes adsorption of an active element, for example iodine, in a natural zeolite for treatment of the skin, especially for feet. This is also an example of a use of hydrophilic zeolites.

EP 0 223 245 A2 describes a dental medication including of a polymer matrix, pharmaceuticals and possible filling material. The filling material can be a hydrophilic zeolite. The filling material increases the compositions mechanical strength and improves the slow release of the pharmaceutical.

JP 63132824 A describes a bandage for absorption of carbon dioxide, hydrogen sulphide and radon. The bandage includes a base of rubber, a carrier of a hydrophilic zeolite and an active substance, for example phenol.

Hydrophobic zeolites on the other hand are normally produced through modification of synthetic, derived hydrophilic zeolites, from which the bigger proportion of Al-ions is eliminated in order to make the zeolite become hydrophobic. This means that the chemical composition of the hydrophobic zeolites in practice approaches pure SiO2.

SE 505 274 can be mentioned as an example of a use of hydrophobic zeolites. It relates itself to use hydrophobic zeolites to eliminate preservatives, for example phenol or cresol, from medicine solution, for example insulin. It is to be notice that the patent intends adsorption of preservatives to the zeolite, not desorption or free release of an active elements from the zeolite.

Another example is SEE 0102265-6, which relates to a system for microbe regulation of a fluid. More specifically the publication relates to an arrangement for the regulation of microbe concentration in a fluid, for example a gas or a fluid. The arrangement includes a hydrophobic zeolite loaded with an element of antiseptic properties. The regulation of microbe concentration on a permanent body, for example the skin, is not described.

In relation to what is known it will be a technology progress to in a simple way achieve a slow release of an active element, for example a disinfection element, for treatment of the skin, for example skin infections.

### SHORT DESCRIPTION OF THE INVENTION

There is a purpose with existing invention to weaken, or to eliminate the disadvantages with the known technology and to achieve a slow release off an active component, for example a disinfection element, for treatment of the skin, for example skin infections.

Through the invention one has found that this purpose can be achieved through that the active component is combined with a hydrophobic zeolite.

More specifically, the invention achieves the use of a hydrophobic zeolite, which contains an active element, in a composition for a non-medical treatment of the skin.

The invention also achieves the use of a hydrophobic zeolite, which contains an active component, for manufacturing of a pharmaceutical preparation for treatment of skin related conditions and diseases.

Further distinguishing features and advantages at the invention are cleared by the subsequent patent claims.

### SHORT BLUEPRINT DESCRIPTION

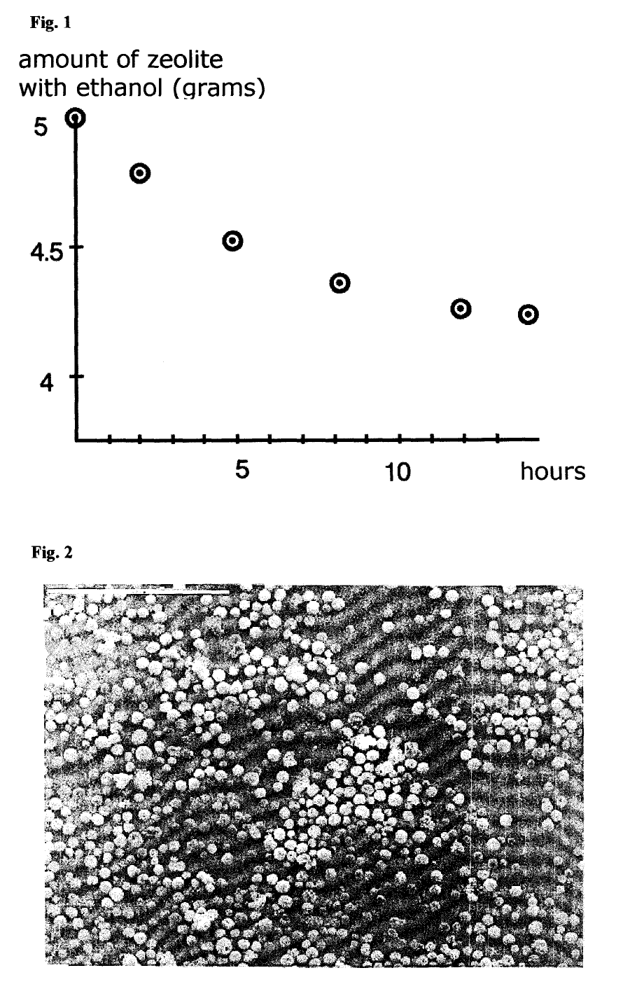

On the attached blueprints, FIG. 1 shows a diagram over how a hydrophobic zeolite, loaded with disinfection elements, release the bacterial killing element related to time. After 12 hours, at 29 degrees C., hydrophobic zeolite Y powder has dropped approximately 70% of its ethanol.

FIG. 2 shows a micro photograph of sintered crystals in form of bulbs, approximately 1 mm in diameter.

### DETAILED DESCRIPTION OF THE INVENTION

The invention is limited to the use of hydrophobic zeolites. This is a necessary and significant distinctive feature of the invention. The use of hydrophilic zeolites does not achieve those purposes and benefits that are aimed at the invention, and will therefore not be covered by the invention.

As been mentioned earlier, hydrophobic zeolites are normally produced through modification of synthetically derived hydrophilic zeolites, from which the bigger proportion of the Al-ions are eliminated in order to make the zeolite become hydrophobic. For instance, hydrophobic zeolite Y is produced schematically through that hydrophilic zeolite Y, with a composition as described above, is treated in cycles with boiling HCl accompanied by heat treatment up to 1100 degrees C. The hydrophobic zeolite Y then gets a composition that lies near to pure SiO2.

The hydrophobic zeolite Y, which is used in the examples below (HSZ−390HUA with LOT No 39UAO901 from TOSOH CORPORATION, TOKYO), has the following chemical composition:
SiO₂/Al₂O₃(mol/mol): 400/1
Na₂O (weight per cent, dry):<0.01
In practice pure SiO₂.

Generally the hydrophobic zeolite is preferably of the type that corresponds to the general basis structure at the network (which then has a negative charge):

[(AlO₂)ₓ(SiO₂)_{y}]

where x and y are integers and y/x>15. Especially preferable is a hydrophobic zeolite with the existing formula, where y/x>100, rather >900.

Hydrophobic zeolites of existing kinds are chosen according to the invention especially from that group that comprises hydrophobic ZSM5, or silicalite, hydrophobic mordenite and hydrophobic zeolite Y, were hydrophobic zeolite Y is speciallily preferred. silicalite has no hydrophilic equivalence.

The pore sizes for stated hydrophobic zeolites normally lies within the area 3-10 Å and the access to the pore system is dimension depending. For hydrophobic zeolite Y and mordenite applies that they have a pore size within the upper area, namely approximately 7,0-7,5 Å, while ZSM5 is a hydrophobic zeolite with pore size of approximately 5,5 Å. The pore system in the hydrophobic zeolite Y and ZSM5 is accessible depending on the three-dimensional arrangement at relevant channels, while mordenite has a one dimensional pore system. The smaller pore size the harder the guest molecule is attached.

For the hydrophobic zeolite applies that it can be used as it is, or in the form of sintered zeolite crystals, in the form of crystals enclosed or suspended in non zeolitic materials, or in the form of bulbs, for example with a diameter of approximately 0,1 mm, as being shown in FIG. 1. It can also be placed on or in other appropriate ways combined with one or several, especially permeable, non zeolitic materials. An example of such non-zeolitic material is ageros.

Possible risks of chemical contamination and biological infection in the use of the hydrophobic zeolite according to the invention can be eliminated through prior treatment off the hydrophobic zeolite. According to an execution form of the invention the hydrophobic zeolite is prior treated, or pre-cleaned through for example, heating to high-temperature, normally over 700 degrees C., preferably over 850 degrees C., and most preferred with the interval 900-1100 degrees C. Another alternative pre-cleaning method involves treatment of the zeolite with so called super critical carbon dioxide.

The chemistry for hydrophobic zeolites is in other areas well known for the professional in the subject and does therefore not need to be described more in detail. Further details concerning hydrophobic zeolites can be received from well known technology.

At the invention the hydrophobic zeolite is combined or “loaded” with an active component, for example a disinfection element, that will be release from the hydrophobic zeolite during slow free release to a permanent substrate, like certain skin, for example the skin on people or animals.

Preferred disinfection elements at the invention are ethanol, iodine, phenol and hydro peroxide. Additional disinfection elements, that are suitable for the use on skin, are well known for the professionals in the subject and can be used at the invention.

The charge of the hydrophobic zeolite with a disinfection element, for example ethanol, can simply be accomplished through that for example ethanol and hydrophobic zeolite are being intermixed in a sealed container. The ethanol then absorbs quickly into the hydrophobic zeolite. Similarly the hydrophobic zeolite can be loaded with iodine. Crystals of iodine is intermixed with the hydrophobic zeolite in a sealed container, where upon the iodine is absorbed in short notice. A more exact charging procedure to load the zeolite is to lead vaporized disinfection elements, for example clean ethanol vapour, over the hydrophobic zeolite, which continuously is weighed so that desired composition is received.

The amount of disinfection elements of which the hydrophobic zeolite is loaded with depends on the specific zeolite that is used (pore size), the specific disinfection element that is used (molecule weight) and that extent of slow free release that are desired. Generally it is preferred that the hydrophobic zeolite is loaded with disinfection elements in an amount of 5-20 weight per cent, more preferred 7-20 weight per cent, counted on the total weight. More special preferred is an amount of 10-20 weight per cent, more preferred 15-20 weight per cent with hydrophobic zeolite Y, and an amount of 5-10 weight per cent, more preferred 7-10 weight per cent with hydrophobic zeolite ZSM5.

The hydrophobic zeolite which is loaded with an active component, especially a disinfection element, for example ethanol, is included as a component in a composition for treatment of the skin. This composition can have the form of a powder, a fluid or a lotion. A powder can for example, be received through intermixing hydrophobic zeolite Y, which is loaded with ethanol, with talk (Mg₃(Si₄O₁₀)(OH)₂) in the weight relation 1:2. A lotion can be received through intermixing hydrophobic zeolite, which is loaded with for example ethanol or iodine, with Vaseline. A fluid can for example, be received through intermixing hydrophobic zeolite Y, which is loaded with ethanol, with olive oil in the weight relation 1:4, or through intermixing hydrophobic zeolite ZSM5, which is loaded with iodine, with paraffine oil. In an additionally examination method hydrophobic zeolite powder, which is loaded with active component, can be placed on a carrier matrix and be included in a bandage.

The existing invention has as well medical as non-medical use. Apart from medical use for the treatment of skin related conditions and diseases, as skin infections and wounds, the invention has a non-medical use as well, for example as a deodorant. One has at the invention also detected that you apart from a bacteria killing effect unexpectedly receive a deodorant effect, i e eliminating undesirable smell, as perspiration smell. At application of hydrophobic zeolite powder, that has been loaded with ethanol, in the armpit you receive an immediate reduction of perspiration smell, which is clear by the examples below.

After describing the existing invention it will now be elucidated additionally through concrete examination examples below, which only are intended to illustrate the invention and may therefore not be considered as limiting for the invention in other meaning from what is clear by the appendix claims. Unless other is stated “weight per cent” means the number weight percent counted on the total weight of hydrophobic zeolite and active component. Unless other is stated “ethanol” means clean ethanol (99,5 weight per cent). As a final point, unless other is stated, 9 persons participated in those tests that are reported below.

### EXAMPLE 1

Hydrophobic zeolite Y was loaded with 16 weight per cent ethanol and its free release of ethanol over the time at a surrounding temperature off 29 degrees C. was studied. The result appears in FIG. 1. Which also appears from FIG. 1, the hydrophobic zeolite had, after approximately 5 hours, given the half amount of its ethanol, and was still active.

In a parallel experiment evaporation of 95% ethanol, not adsorbed to any hydrophobic zeolite, was measured. One gram of this ethanol had after 8 minutes been reduced through evaporation to 0,5 grams, i e to half the amount. This is a drastic difference compared with ethanol which is adsorbed to hydrophobic zeolite. If the experiment had been executed with 99.5% ethanol instead of 95% it would presumably have been even smaller. Through the hydrophobic zeolite composition the skin is exposed to ethanol vapour during 50 times's longer time as in a normal sponging.

The mentioned hydrophobic zeolite loaded with ethanol gives a vapour that is near 100% ethanol in composition. The ethanol vapour can aggressively attack skin bacteria's in their hydrophobic environment during relatively long time.

### EXAMPLE 2

### Arm Perspiration and Hydrophobic Zeolite Powder

Hydrophobic zeolite Y was loaded with ethanol in an amount of 16 weight per cent and was intermixed with talc in the weight ratio 1:2. The powder mixture was located in armpits once daily. In this case it was observed that the perspiration smell was reduced or disappeared. After ending the treatment the effect left in up to three days.

### EXAMPLE 3

### Arm Perspiration and Hydrophobic Zeolite Fluid

Hydrophobic zeolite Y was loaded with ethanol in an amount of 16 weight per cent and was intermixed with olive oil in the weight relation 1:4. The fluid mixture was located in armpits with a roller of normal type once daily. No perfume was added. In this case four persons were included in the test team. All people reported that the deodorant with the hydrophobic zeolite was just as effective or almost as effective as that commercial deodorant normally used. After ending the treatment the effect left in up to three days.

### EXAMPLE 4

### Small Wounds

Above mentioned hydrophobic zeolite Y containing 16 weight per cent ethanol was mixed with Vaseline until appropriate ointments consistency was received. The completed ointment contained 3-5 weight per cent ethanol. Common small wounds were treated and a process of healing started immediately. The ointment seemed to prevent arise of infection phase in all those cases that were studied.

### EXAMPLE 5

Changes with Age 1

Typical changes with age in face, especially cheeks and around the hairline in pan gives small wounds that heal very slow. One person in the test team had several years of problems. Above mentioned ointment according to examples 4 gave proper start of healing in two days. After 8-10 days treatment permanent healing had been received except in two cases (see below).

### EXAMPLE 6

Changes with Age 2

Bilaterally the person in the test team with several years of problems had two several years old wounds on each cheek. Above mentioned ointment according to examples 4 gave a good start of healing in two days, and after two weeks treatment permanent healing seemed too been received. However the wounds came back after additional two weeks. New ointment was produced with 3 weight parts Vaseline mixed with 2 weight parts of the hydrophobic zeolite ZSM5, which was loaded with 10 weight per cent iodine. A dramatic improvement was received, and after as early as some hours later the process of healing where well under way. After three days it all seemed to be healed. Still, the treatment was continued seven more days. Permanent healing seemed to have been received 30 days after completed treatment.

General applies that treatment with hydrophobic zeolite loaded with ethanol took place twice a day. Treatment with hydrophobic iodine loaded zeolite took place once a day.

1-13. (canceled)

14. A. composition for non-medical treatment of the skin which comprises an active component and a hydrophobic zeolite, that contains and gives a slow release of the active component.

15. The composition according to claim 14 being a deodorant composition.

16. The composition according to claim 14 which comprises a hydrophobic zeolite, that contains and gives a slow release of an active component, for the request of a medicine for treatment of skin related conditions and diseases.

17. The composition according to claim 16 where the skin related conditions and diseases are skin infections.

18. The composition according to claim 16 whereupon the active component is a disinfection element.

19. The composition according to claim 14, wherein the active component is at least one member selected from the group consisting of ethanol, iodine, phenol, cresol and hydrogen peroxide.

20. The composition according to claim 14, wherein the composition contains 5-20 weight per cent active component.

21. The composition according to claim 20, wherein the composition contains 7-20 weight per cent active component.

22. The composition according to claim 14, wherein the hydrophobic zeolite has a basis structure with the general formula
[(AlO2)x(SiO2)y] where x and y are integers and y/x>15, preferably >100, more preferred >900.

23. The composition according to claim 14, wherein the hydrophobic zeolite is selected from the group consisting of silicalite or hydrophobic ZMS-5, hydrophobic mordenite and hydrophobic zeolite Y.

24. The composition according to claim 20, wherein the hydrophobic zeolite is hydrophobic zeolite Y.

25. The composition according to claim 14, wherein the hydrophobic zeolite is vapour cleaned through heating to a temperature in excess of 700 degrees C.

26. The composition according to claim 14, wherein the composition has the form of a powder, a fluid, ointment or is included in a bandage.

28. The composition according to claim 17, whereupon the active component is a disinfection element.

29. The composition according to claim 16, wherein the active component is at least one member selected from the group consisting of ethanol, iodine, phenol, cresol and hydrogen peroxide.

30. The composition according to claim 16, wherein composition contains 5-20 weight per cent active component.

31. The composition according to claim 16, wherein the hydrophobic zeolite has a basis structure with the general formula
[(AlO2)x(SiO2)y] where x and y are integers and y/x>15, preferably >100, more preferred >900.

32. The composition according to claim 16, wherein the hydrophobic zeolite is selected from the group consisting of silicalite or hydrophobic ZMS-5, hydrophobic mordenite and hydrophobic zeolite Y.

33. The composition according to claim 16, wherein the hydrophobic zeolite is vapour cleaned through heating to a temperature in excess of 700 degrees C.

34. The composition according to claim 16, wherein the composition has the form of a powder, a fluid, ointment or is included in a bandage.
